(19) United States
(12) Patent Application Publication
(10) Pub. No.: US 2005/0228364 A1
(43) Pub. Date: Oct. 13, 2005

(54) Tunneler device

(76) Inventor: Richard Braga, Taunton, MA (US)

Correspondence Address: Mark S. Leonardo, Esq.;Brown Rudnick Berlack Israels LLP, One Financial Center, Boston, MA, 02111, US

(21) Appl. No.: 10/997,424

(22) Filed: Nov. 24, 2004

Related U.S. Application Data
(60) Provisional application No. 60/561,010, filed on Apr. 9, 2004.

(51) Int. Cl. A61B 17/00

(52) U.S. Cl. 606/1; 403/377

(57) ABSTRACT

A tunneler is provided that includes a first end portion configured for insertion into a lumen of a catheter. The tunneler also includes a second end portion opposite the first end portion and a sheath member disposed between the first and second end portions. The sheath member includes a plurality of inner diameters that increase from one end to the other end thereof. Alternatively, the plurality of inner diameters taper from one end to the other end thereof. The inner diameter of the sheath member engages an outer surface of the catheter to create an interference fit therebetween. The interference fit increases a catheter pull-off force. A method of using the tunneler is also disclosed.

### RELATED APPLICATION INFORMATION

This patent application claims priority from U.S. Provisional Application No. 60/561,010, filed with the United States Patent and Trademark Office on Apr. 9, 2004, the entire contents of which is incorporated herein by reference.

### BACKGROUND

1. Technical Field

The present disclosure relates generally to tunneling devices for use with medical catheter apparatuses, and more particularly to a tunneling device for aiding medical catheter insertion.

2. Background of the Related Art

Some known catheters are tubular, flexible medical devices for administration of fluids (i.e., withdrawal, introduction and the like) within veins, cavities, ducts and vessels of a body.

These catheter devices may be employed for administration of fluids that include the simultaneous introduction and withdrawal of fluid for applications such as, surgery, treatment, diagnosis and the like. In one particular hemodialysis application, blood is withdrawn from a blood vessel for treatment by an artificial kidney device and the treated blood is introduced back into the blood vessel.

Various known catheter devices have been employed for simultaneous withdrawal and introduction of fluid with a body. These devices may utilize multiple lumens, such as dual lumen catheters that facilitate bidirectional fluid flow whereby one lumen performs withdrawal of blood and the other lumen introduces treated blood to the vessel. During an exemplary hemodialysis procedure, a multiple lumen catheter is inserted into a body and blood is withdrawn through an arterial lumen of the catheter. This blood is supplied to a hemodialysis unit that dialyzes or cleans the blood to remove waste and excess water. The dialyzed blood is returned to the patient through a venous lumen of the catheter. Typically, the venous lumen is separated from the arterial lumen by an inner catheter wall known as a septum.

A tunneling device or “tunneler” may be used to facilitate catheter placement in the patient. For optimal performance during dialysis treatment, the catheter tips, both in-flow and out-flow, may be placed in close proximity to the heart. Typically, medical personnel use either a single, double or multiple lumen catheter.

While single, double or multiple lumen catheters allow for venous insertion of the catheter into the desired vein, accuracy of catheter tip placement is always paramount. Due to differences among patients, optimal tip position varies from patient to patient. Non-optimal tip position may significantly lower flow values, resulting in less effective dialysis treatment.

For current catheters, a physician must make an estimate regarding the appropriate catheter tube length prior to beginning the procedure of catheterization. Then, a subcutaneous tunnel is made from the preferred end position of the hub assembly, for example, away from the neck of the patient in order to allow for more convenient access to the dialysis treatment equipment. The catheter tube is then tunneled forwardly into the patient's vein. The initial estimate and subsequent forward tunneling may result in less than optimal tip placement.

One problem associated with the use of catheters, for example, hemodialysis catheters, is the use of separate venous insertions, for example, two tunnels and two of each accessory instrument used for the procedure. Therefore, there is increased surgical time required to place the catheters and two wound entry sites that doubles the risk of post-surgical infection.

There is a need in the art for a tunneler that improves catheter placement.

### SUMMARY

In one particular embodiment, a tunneling device is provided having a first end portion configured for insertion into a lumen of a catheter. The tunneler also includes a second end portion opposite the first end portion and a sheath member disposed between the first and second end portions. The sheath member has a plurality of inner diameters. The plurality of inner diameters increase in steps from one end of the sheath member to the other end thereof. Alternatively, the plurality of inner diameters taper from an end of the sheath member to the other end thereof. The stepped inner diameters of the sheath member engage an outer surface of the catheter to create an interference fit therebetween. The interference fit increases a catheter pull-off force.

In an alternate embodiment, the tunneler comprises a distal portion having at least one insert member configured for insertion into a lumen at a distal end of the catheter. The insert member has at least one projection for engaging an inner surface of the catheter lumen. The catheter is held onto the insert member by an interference fit therebetween created by an expansion of catheter material around the at least one projection. The tunneler also includes a proximal portion of metal construction and opposite the distal portion. This proximal portion has a blunt surface configured for subcutaneous tunneling. The sheath member is disposed between the distal and proximal portions. The sheath member has a portion with a tapered configuration and another portion with a rounded straight edge. The sheath member has an annular interior surface and a plurality of inner diameters. The plurality of inner diameters increase in steps from the tapered portion of the sheath member to the rounded straight edge portion thereof. The interior surface of the sheath member has stepped diameters that engage an outer surface of the catheter to create an interference fit therebetween. The interference fit between the interior surface of the sheath member and the outer surface of the catheter increases a force required to detach the catheter from the insert member.

The present invention also provides a method for inserting a catheter assembly into an area to be catherized. The method involves making an incision near the area to be catherized. The sheath member is slid back to expose the first end of the tunneler. Thereafter, the catheter lumen is slid over the first end of the tunneler. The sheath member of plural diameters is slid back over the distal portion of the catheter until it stops firmly covering the distal portion of the catheter and the first end of the tunneler. The second end of the tunneler is thereafter inserted into the area to be catherized, to create a subcutaneous tunnel and route the distal portion of the catheter through the tunnel. Thereafter, the sheath member is slid back to expose the distal portion of the catheter which is then removed from the first end of the tunneler.

### BRIEF DESCRIPTION OF THE DRAWINGS

The invention will be apparent from the description herein and the accompanying drawings, in which like reference characters refer to the same parts throughout the different views.

FIG. 1A is a perspective view of an example embodiment of a tunneler in accordance with the principles of the present disclosure;

FIG. 1B is an enlarged callout view of the tunneler shown in FIG. 1A;

FIG. 2 is an enlarged cross-sectional view of the sheath of the tunneler shown in FIG. 1A;

FIG. 3A is an enlarged cross-sectional view of the tunneler shown in FIG. 1A as attached to a catheter device; and

FIG. 3B is a cross-sectional view of the tunneler-catheter assembly shown in FIG. 3A.

### DETAILED DESCRIPTION OF THE EXEMPLARY EMBODIMENTS

The various embodiments of the present invention may be used with medical catheters and related medical devices including the use and insertion of medical catheters for the administration of fluids (i.e., withdrawal, introduction and the like) with the body of a subject and more particularly, in terms of a catheter that facilitates placement with a body vessel. The tunneler may be configured to reduce trauma and prevent patient complications. It is envisioned that the tunneler of the present disclosure may be employed with a range of catheters, such as, for example, hemodialysis, peritoneal, infusion, PICC, CVC, port and catheter applications including surgical, diagnostic and related treatments of diseases and body ailments of a subject. It is further envisioned that the principles relating to the tunneler disclosed include use with various catheter related procedures, such as, for example, hemodialysis, cardiac, abdominal, urinary, intestinal and the like in chronic and acute applications.

In the discussion herein that follows, the term “proximal” will refer to the portion of a structure that is closer to a practitioner, while the term “distal” will refer to the portion that is further from the practitioner. As used herein, the term “subject” refers to a human patient or other animal. According to the present disclosure, the term “practitioner” refers to a doctor, nurse or other care provider and may include support personnel.

The following discussion herein includes a description of the tunneler, in accordance with the principles of the present disclosure. Reference will now be made in detail to the exemplary embodiments of the disclosure, which are illustrated in the accompanying figures.

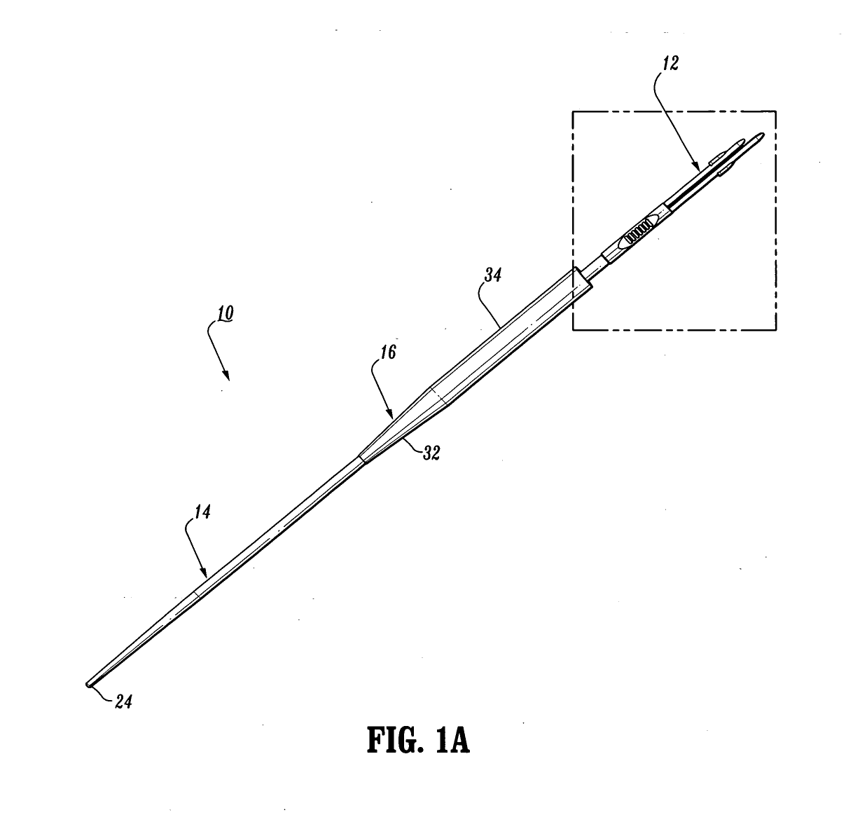

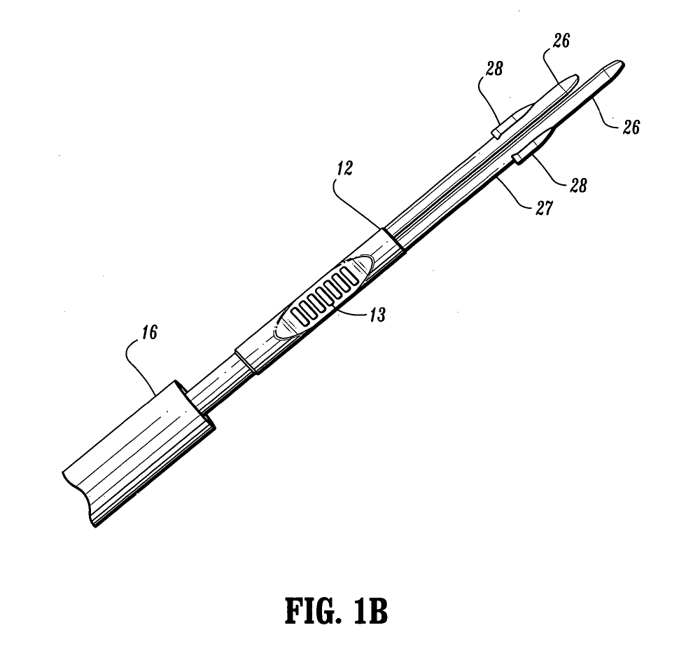

With reference to FIGS. 1-3, an example embodiment of the tunneler 10 is configured for use as a catheter tunneler to aid in the insertion of a single, double or multiple lumen hemodialysis catheters into a subject.

The tunneler 10 includes a distal end 12 configured for engagement or coupling with a catheter. End portion 12 is made of plastic material such as nylon, although metal or other materials may be used. In the illustrative embodiment, distal end 12 is bifurcated to define two separate insert members, bifurcations or tines 26 for inserting into the catheter lumens 38. However, distal end 12, instead of being bifurcated, may instead define a single or multiple insert members. Each of the tines 26 include one or more projections, ribs or nodes 28 along a circumferential surface 27 thereof for engaging the lumen surfaces 30.

Tunneler 10 also includes proximal end 14 configured for tunneling through subcutaneous skin of a subject. End portion 14 is tapered and constructed from metal, although plastic or other materials may be used, and includes tip 24 which may be blunted to facilitate tunneling without causing tissue trauma. Tunneler 10 further includes a sheath member 16. Sheath 16 is slidably disposed between distal and proximal ends 12, 14 of tunneler 10. The sheath has a tapered end portion 32 oriented toward the proximal end 14 of the tunneler 10 and a straight portion 34 oriented toward the tunneler's distal end 12 for receiving the tip portion 20 of the catheter.

Operation of the tunneler 10 according to an example embodiment may include the following actions; however, the present disclosure is not limited to these actions or order of occurrence. A practitioner using the tunneler 10 may slide the sheath 16 away from the tunneler's distal end 12 to expose tines 26. The practitioner then may secure catheter tip 20 to tunneler 10 by inserting tines 26 into catheter lumens 38. More specifically, the practitioner may grasp the tunneler's ribbed finger pad 13 with thumb and forefinger and insert tines 26 into lumens 38 simultaneously. Alternatively, for example, the longer tine 26 may be first inserted in one lumen 38, and the shorter tine 26 is thereafter inserted into the other lumen 38. Having thus secured catheter and tunneler 10, the practitioner slides sheath 16 back over catheter tip 20 until it stops firmly covering tip 20 and tines 26. The assembly of tunneler 10 and catheter is then utilized to tunnel the catheter subcutaneously into the subject. More specifically, the practitioner inserts the tunneler's proximal end 14 through a first incision made in the chest, neck or other body site of the subject. Tunneler 10 is then manipulated up and through the subcutaneous skin (i.e., tunneled) to exit out at a second incision made in the subject's neck, chest or other body site. By way of example, the tunnel from a chest insertion site to a neck exits site may be 3 ′-5 in length. The catheter tip 20 is pulled through the tunnel, leaving the proximal end of the catheter (not shown) at the incision site. Once the catheter is pulled through the tunnel, the practitioner may slide sheath 16 back to expose catheter tip 20 and catheter tip 20 is disengaged from tunneler 10 by removing tines 26 from lumens 38.

In the above example of operation, it is desired on the one hand that the practitioner be able to connect together the catheter and tunneler prior to tunneling, as well as separate and remove the catheter and tunneler from each other after tunneling. During subcutaneous tunneling, on the other hand, it is highly desired that the catheter and tunneler remain sufficiently connected so not to separate or pull-off from each other.

Accordingly, the present disclosure reduces the catheter-to-tunneler insertion connection and separation forces (i.e., the forces required to connect or separate catheter tip 20 from tunneler end 12, before, during and after tunneling). Moreover, the catheter tip 20 and tunneler end 12 pull-off force is increased during tunneling, such that catheter tip 20 stays secured to tunneler end 12 while the catheter-tunneler assembly travels subcutaneously in the subject.

Reduction of the catheter-to-tunneler insertion and removal forces is provided at least in part as follows. As the practitioner inserts tunneler tines 26 into catheter lumens 38, tine surfaces 27 engage lumen surfaces 30 which expand around nodes 28. In this manner, catheter tip 20 is held onto tunneler 10 by a first interference fit 40 between nodes 28 and lumen surface 30. This first interference fit 40, however, still allows the practitioner to connect and separate the catheter and tunneler 10 prior to and following tunneling.

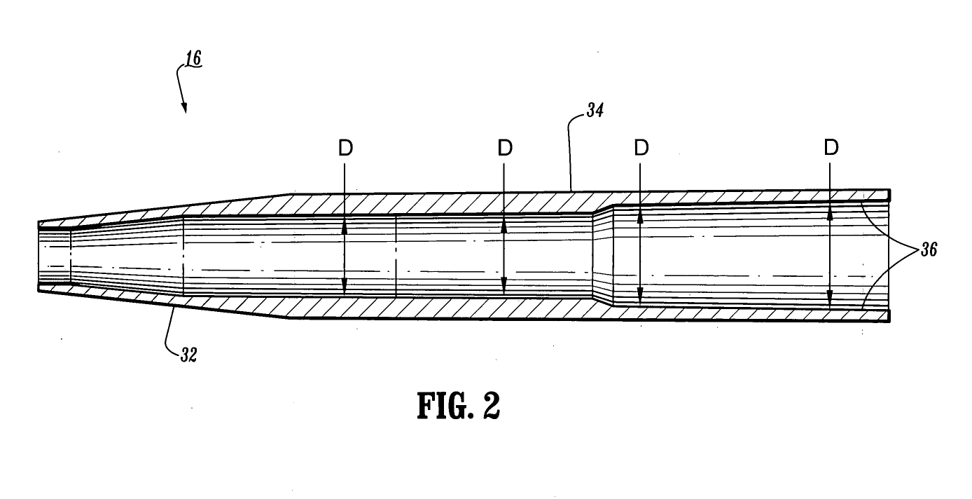

To increase the catheter pull-off force during tunneling, however, the present disclosure provides for additional interference fits 40 for preventing separation of the catheter and tunneler 10 while the catheter-tunneler assembly travels subcutaneously in the subject. With reference to FIG. 2, sheath 16 has a plurality of inner diameters D. In the illustrative embodiment, diameters D increase in steps from one end 32 of sheath 16 to another end 34 thereof. Alternatively, diameters D may increase, decrease or alternate in a tapered fashion.

To create the stepped diameters 18, added wall thickness 50 is provided to the interior of sheath 16 by shifting the internal lead-in angle α 54 back along the sheath's central axis 52. This geometry results in a second interference fit 56, that is, between catheter tip 20 and sheath 16, which increases the catheter pull-off force. It is contemplated within the present disclosure that a plurality of interference fits may be provided between the sheath 16 and the tines 26 and nodes 28 and between the sheath 16 and the catheter (e.g., catheter end 20).

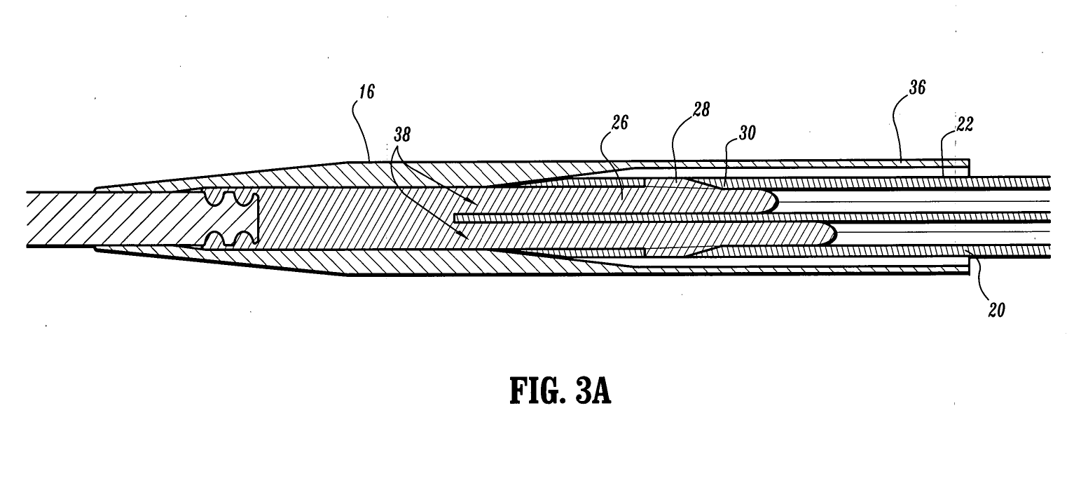

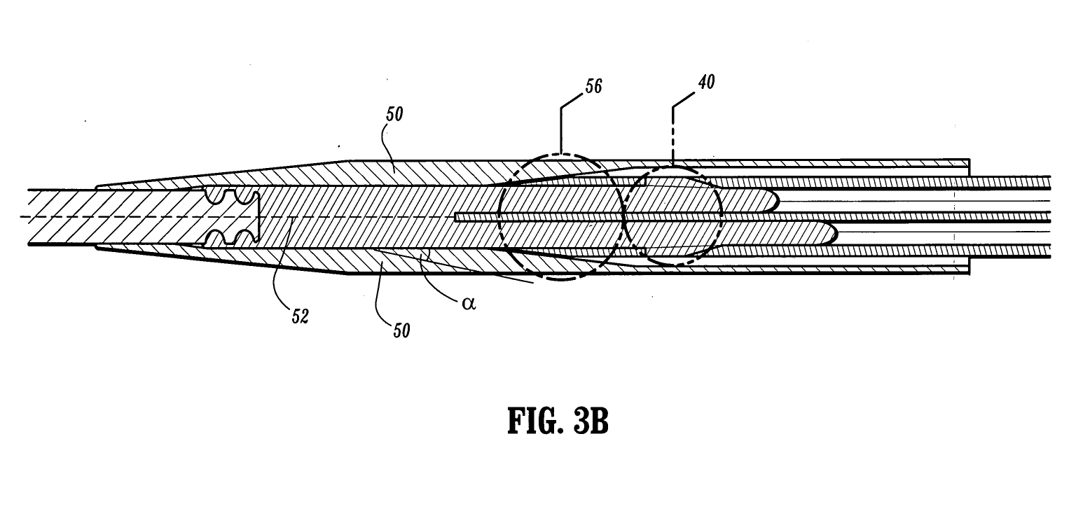

With reference to FIG. 3A and 3B, when sheath 16 is slid over catheter tip 20, the sheath's interior wall 36 engages outer catheter surface 22 to create the second interference fit 56 therebetween. This second interference fit 56 (i.e., between sheath 16 and the catheter) adds to the first interference fit (i.e., between nodes 28 and the catheter), so to increase the pull-off force and prevent separation of catheter and tunneler during tunneling.

After tunneling, the practitioner may slide sheath 16 away from catheter tip 20, so that second interference fit 56 is no longer present. Since only the first interference fit 40 (between nodes 28 and catheter 20) now remains, the practitioner can separate catheter and tunneler 10, concluding the tunneling procedure.

Alternate methods of tunneling using the tunneler of the present disclosure are known, for example, U.S. Pat. No. 6,638,242 to Wilson et al., which is incorporated herein in its entirety, discloses various manners of subcutaneously tunneling through skin that can be employed and enhanced using the tunneler of the present disclosure.

The present invention has been described by way of example, and modifications and variations of the exemplary embodiments will suggest themselves to skilled artisans in this field without departing from the spirit of the invention. Features and characteristics of the above-described embodiments may be used in combination. The preferred embodiments are merely illustrative and should not be considered restrictive in any way. The scope of the invention is to be measured by the appended claims, rather than the preceding description, and all variations and equivalents that fall within the range of the claims are intended to be embraced therein.

1. A coupler for use with a catheter, comprising:
at least one insertion device, elongated along a first dimension, having a first end for inserting into a lumen of the catheter;
at least one projection, located on the insertion device and extending at least partially in a second dimension, perpendicular to the first dimension; and
a sheath, movable along the first dimension and sized to locate the catheter between the sheath and the at least one insertion device, an inner surface of the sheath capable of forming an interference fit with the catheter.

2. The coupler of claim 1, wherein the sheath has an inner surface having a plurality of inner dimensions.

3. The coupler of claim 2, wherein each of the plurality of inner dimensions is progressively smaller from a first inner dimension located proximate to the first end of the at least one insertion device.

4. The coupler of claim 2, wherein each of the plurality of inner dimensions forms a step along the inner surface.

5. The coupler of claim 1, wherein the sheath has an inner surface having a plurality of inner diameters.

6. The coupler of claim 5, wherein each of the plurality of inner diameters is progressively smaller from a first inner diameter located proximate to the first end of the at least one insertion device.

7. The coupler of claim 5, wherein each of the plurality of inner diameters forms a step along the inner surface.

8. A tunneling device, comprising:
an elongated body having a first end and a second end, opposite the first end;
at the first end of the body, at least one insertion device, elongated along a first dimension, having a first end for inserting into a lumen of the catheter;
at least one projection, located on the insertion device and extending at least partially in a second dimension, perpendicular to the first dimension;
a sheath, movable along the first dimension and sized to locate the catheter between the sheath and the at least one insertion device, an inner surface of the sheath locatable proximate to the at least one projection; and
at the second end of the body, a tunneling tip adapted to tunnel through subcutaneous tissue.

9. A tunneling device comprising:
a first end portion configured for insertion into a lumen of a catheter;
a second end portion opposite the first end portion; and
a sheath member disposed between the first and second end portions, the sheath member having a plurality of inner diameters.

10. The tunneling device of claim 9, wherein the plurality of inner diameters increase in steps from one end of the sheath member to another end thereof.

11. The tunneling device of claim 9, wherein at least one of the plurality of stepped inner diameters of the sheath member engage an outer surface of the catheter to create an interference fit therebetween.

12. The tunneling device of claim 11, wherein the interference fit increases a catheter pull-off force.

13. The tunneling device of claim 10, wherein the plurality of inner diameters taper from the one end of the sheath member to the other end thereof.

14. A tunneling device, the device comprising:
a distal portion having at least one insert member configured for insertion into a catheter lumen at a first end of a catheter, the insert member having at least one projection for engaging an inner surface of the catheter lumen,
a proximal portion opposite the distal portion, the proximal portion being configured for subcutaneous tunneling; and
a sheath member disposed between the distal and proximal portions, the sheath member having a tapered portion and a plurality of inner diameters.

15. The tunneling device of claim 14, wherein the catheter is held onto the at least one insert member by a first interference fit between the catheter and the at least one insert member.

16. The tunneling device of claim 14, wherein the plurality of inner diameters increase in steps from the tapered portion of the sheath member to a non-tapered portion thereof.

17. The tunneling device of claim 14, wherein an interior surface of the sheath member engages an outer surface of the catheter to create a second interference fit therebetween.

18. The tunneling device of claim 17, wherein the second interference fit between the interior surface of the sheath member and the outer surface of the catheter increases a force required to detach the catheter from the at least one insert member.

19. The tunneling device of claim 14, wherein the plurality of inner diameters taper from the tapered portion of the sheath member to a non-tapered portion thereof.

20. A method of using a tunneler, the method comprising the steps of:
providing a tunneler having a first end configured for insertion into at least one lumen of a catheter, a second end opposite the first end, and a sheath member disposed therebetween, the sheath member having a plurality of inner diameters;
attaching the tunneler to the catheter; and
sliding the sheath member over a portion of the catheter to create an interference fit between at least one of the inner diameters and the portion of the catheter.

21. The method of claim 20, wherein the diameter of the plurality of inner diameters increase in the direction from an end of the sheath member to another end thereof.

22. The method of claim 20, wherein the interference fit between the at least one of the inner diameters and the portion of the catheter increases a catheter pull-off force.

23. The method of claim 20, wherein the plurality of inner diameters taper from an end of the sheath member to another end thereof.
